(19) United States
(12) Patent Application Publication
(10) Pub. No.: US 2005/0202510 A1
(43) Pub. Date: Sep. 15, 2005

(54) Method for identifying a site of protein-protein interaction for the rational design of short peptides that interfere with that interaction

(75) Inventors: Relly Brandman, Stanford, CA (US); Daria Mochly-Rosen, Menlo Park, CA (US)

Correspondence Address: BOZICEVIC, FIELD & FRANCIS LLP, 1900 UNIVERSITY AVENUE, SUITE 200, EAST PALO ALTO, CA, 94303, US

(73) Assignee: The Board of Trustees of the Leland Stanford Junior University

(21) Appl. No.: 11/059,482

(22) Filed: Feb. 15, 2005

Related U.S. Application Data
(60) Provisional application No. 60/547,702, filed on Feb. 24, 2004.

(51) Int. Cl. G01N 33/53; G06F 19/00; G01N 33/48; G01N 33/50

(52) U.S. Cl. 435/7.1; 702/19

(57) ABSTRACT

The invention provides a method for identifying a site of protein-protein interaction in a polypeptide. In general, the method involves calculating the difference in property scores between amino acids of a corresponding pair of amino acids on two homologous polypeptides, and identifying a window of contiguous amino acids that have a significant difference in property scores. The contiguous amino acids are predicted to be sites of protein-protein interactions. The invention provides computer systems for performing the methods. The subject methods and computer systems find application in identifying modulators of protein-protein interactions that can serve as inhibitors or activators of the protein from which it was derived, and, as such, find use in a variety or medical and research applications, including drug discovery.

### FIELD OF THE INVENTION

This invention relates to software tools for the analysis of proteins, particularly for predicting sites of protein-protein interactions.

### BACKGROUND OF THE INVENTION

Protein-protein interactions are involved in many aspects of cell biology, and, as such, are of intense interest to the research and medical communities. It is believed that by identifying and understanding protein-protein interactions, drugs that modulate those interactions may be easier developed. Since drugs that mimic a site of protein-protein interaction often may be used to inhibit protein-protein interactions in a cell, methods for the identification of sites of protein-protein interaction are of particular interest (Veselovsky et al., J Mol Recognit. (2002) 15:405-22; Souroujon et al., Nat Biotechnol. (1998) 16:919-24). Accordingly, there is a great need for convenient, accurate, and rapid tools to identify and selectively regulate sites of protein-protein interaction.

In an attempt to meet this need, a number of different types of methods have been developed. Such methods include biochemistry-based assays, e.g., co-immunoprecipitation and affinity assays, high throughput assays using a library of small molecules and a simple in vitro interaction assay such as ELISA (Vassilev et al., Science (2004) 0: 10924721-0) in vivo assays, e.g., “two hybrid” assays, and bioinformatics methods (e.g., those described in Ng et al., Bioinformatics (2003) 19:923-9). However, all of these methods require prior knowledge of the identity of the proteins that are interacting. Accordingly, conventional methods are not practical, especially when the identity of the protein that binds to a protein of interest is not known. In addition, computational modeling and prediction of protein-protein interactions almost always requires prior knowledge of protein structure. Because the structure of most proteins is not known, and because it is thought that reliable modeling of structure requires the existence of a known structure from a close homologue, many proteins are not candidates for these prediction methods. Finally, many methods of identifying sites of protein-protein interactions do not distinguish between very close homologues, for example different isozymes in an enzyme family. Thus any modulation at these predicted sites may result in a non specific effect.

In other words, while sites of protein-protein interaction can be predicted in a protein of interest using any of the above methods, the methods themselves cannot usually be performed unless a binding partner for the protein of interest has been identified. Since most methods for identifying proteins that bind to each other require a considerable amount of effort and are generally error prone, mapping sites of protein-protein interaction usually requires a vast amount of work. In addition, experimental methods often reveal only high affinity interactions, and as such are prone to miss an important and large subset of protein-protein interactions, transient interactions (for example, kinase-substrate interactions). Furthermore, computational modeling tools require previous knowledge of protein structure and often result in prediction of sites that are common to more than one protein. Finally, existing methods only identify intermolecular interactions and cannot predict sites of intramolecular interactions.

Current methods of discovering peptide modulators of protein-protein interactions involve screening either random or biased peptide libraries. For example, random libraries of all possible peptides of a certain length can be screened, for example by phage display (Scott et al, Science (1990) 249:386-390). An example of a biased peptide screen would be choosing only peptides that have particular key amino acids that are known to be involved in a protein-protein interaction (Fantl et al, Cell (1992) 69:413-423). These methods, however, are costly and time-intensive because they involve screening many peptides before a modulator is found. In the case of biased screens, prior information about the protein-protein interaction site is required to limit the peptides tested. Importantly, these methods do not ensure that peptides found to modulate protein-protein interactions will be specific. Finally, these methods of discovery may lead to potential biologically active protein-protein interaction modulators, however they do not predict protein-protein interaction sites.

There are also numerous computational tools available for identifying binding sites. However, these methods typically rely on molecular modeling, are computationally intensive, and not generally useable without a significant amount of structural information. For example, the POCKET program (Levitt et al., J. Mol. Graphics (1992) 10: 229-234), which is a computer graphics program for identifying and displaying protein cavities and their surrounding amino acids, can be used to identify exposed surface area and small pockets, regions that have the potential to be binding sites. The GRID potential (Goodford, J. Med. Chem. (1985) 28:849-857) calculates regions within the protein that have a high affinity for different types of “probes” using a semi-empirical potential. Thus, it can be used to compute favorable interaction sites for different atoms or functional groups within the binding site of a target protein. The DOCK program (Kuntz et al., J. Mol. Biol. (1982) 161:269-288) is a geometric approach to molecular interactions that docks every molecule in a database of small molecules into a binding site of a target protein and reports on the best hits that it finds. The MSI Ludi program (Bohm, J. Comput. Aided Mol. Des. (1992) 6:61-78) is a method for de novo design of enzyme inhibitors that can perform fragment searches to identify molecular fragments that will most readily interact with a target enzyme. The SiteID program (Tripos Inc., 1699 South Hanley Rd., St. Louis, Mo., 63144, USA), VOIDOO (Kleywegt et al., Acta Cryatallogr. (1994) D50:178-185), HOLE (Smart et al., Biophys. J. (1993) 65:2455-2460) and SURFNET algorithm (Laskowski J. Mol. Graph. (1995) 13:323-330) are other examples of such programs.

Some computational methods incorporate amino acid variability over evolution to predict functionally important sites. Sites that are evolutionarily conserved are predicted to be functionally important. If these sites lie on protein surfaces, they are inferred to be involved in protein-protein interactions. These methods depend on the knowledge or prediction of protein structure. For example, Lichtarge et al., (J. Mol. Biol. 257, 342-358), have developed a method which identifies patches on the three dimensional protein structure and looks for regions that are conserved over evolution. These regions are predicted to involve protein-protein interactions, however since they do not correspond to a simple polypeptide chain, a short peptide that will mimic the site and will interfere with these protein-protein interaction can not be designed based on this information.

Accordingly, while there is a great need for convenient, accurate, and rapid tools to identify sites of protein-protein interaction, particularly for proteins that have uncharacterized binding partners, such tools are not yet available. This invention, however, meets these needs, and others.

Literature of interest includes: Schechtman et al., Methods Enzymol. (2002) 345:470-89; Souroujon et al., Nat Biotechnol. (1998) 16:919-24; Chen et al., Proc Natl Acad Sci USA. (2001) 98:11114-9; Stebbins et al., J Biol Chem. (2001) 276:29644-50; Kawashima et al., Nucleic Acids Res. (2000) 28:374; Mendez et al., Proteins. (2003) 52:51-67; Jones, J Mol Biol. (1997) 272:133-43; Ng, Bioinformatics (2003)19:923-9; Dandekar et al., Trends Biochem Sci. (1998) 23:324-8; Casari et al., Nat Struct Biol. (1995) 2:171-8; Jameson et al., Comput Appl Biosci. (1998) 4:181-6; Kolaskar, FEBS Lett. (1990) 276:172-4; and Lichtarge et al., J. Mol. Biol. 257, 342-358; published U.S. patent applications Nos. 20030180803 and 20030130827; and PCT publications WO98/54665 and WO01/16862.

### SUMMARY OF THE INVENTION

The invention provides an automated method for both identifying and modulating a site of protein-protein interaction in a protein. In general, the method comprises calculating the difference in property scores between amino acids of a corresponding pair of amino acids on two homologous polypeptides, and identifying at least six contiguous amino acids that have a significant difference in property scores. The contiguous amino acids are predicted to be sites of protein-protein interaction. The invention provides computer systems for performing the methods. The subject methods and computer systems find application in identifying inhibitors of protein-protein interactions, and, as such, find use in a variety or medical and research applications, including drug discovery.

The subject methods do not depend on a known 3D structure (although when available, it can be used to augment the subject methods) or experimental data, as do conventional computational methods for identifying sites of protein-protein interaction.

In fact, the invention described herein is the only automated method that is able to predict protein binding sites on two homologous proteins, for example isoenzymes. Further, the subject methods do not depend on information about the identity of the binding partner, which allows the methods to be applied to a wider range of protein families.

Further, unlike most computational methods developed to predict antigenic peptides, the subject methods may be successfully used to predict sites of protein-protein interaction in both soluble and insoluble proteins.

The subject methods are able to identify both intramolecular interactions and intermolecular interactions. Existing methods of predicting sites of protein-protein interaction identify only intermolecular protein interactions. Often a protein has different structural conformations in its active and inactive forms, and intramolecular interactions are autoinhibitory. Interfering with intramolecular interactions can therefore cause a protein to be more stable in its active conformation. Thus when designing drugs based on the sites of protein-protein interactions, the subject invention may be used to predict both activators and inhibitors of proteins. Other methods are only able to predict inhibitors.

Unlike any other prediction method, the subject invention not only predicts protein-protein interaction sites but also designs biologically active peptides to modulate these sites. These peptides or mimetics can be used as drugs or drug precursors (drug leads) that work by activating or inhibiting only a specific protein of interest.

In summary, the subject methods may be applied to a larger number of proteins than existing computational methods because the methods are able to make predictions based on very limited data (although, when available, additional data may be used to supplement the subject methods). The subject methods are also more specific than other methods, allowing predicted peptides to act selectively on individual members of families of homologous proteins. Finally, in some embodiments, the outcome output of the subject methods is a biologically active peptide that may be used to inhibit protein-protein interactions, rather than a theoretical prediction of the location(s) of protein-protein interaction(s). Pharmacological agents predicted by the subject methods are ready to be synthesized and used, thus bridging the gap between a theoretical prediction and a drug.

### BRIEF DESCRIPTION OF THE DRAWINGS

FIG. 1 is flow diagram that diagrammatically shows an exemplary embodiment of the invention.

FIG. 2 is a block diagram showing a computer system for use in the subject methods.

### DETAILED DESCRIPTION OF THE INVENTION

The invention provides a method for identifying a site of protein-protein interaction in a polypeptide. In general, the method includes calculating the difference in property scores between amino acids of a corresponding pair of amino acids on two homologous polypeptides, and identifying at least six contiguous amino acids that have a significant difference in property scores. The contiguous amino acids are predicted to be sites of protein-protein interaction. The invention provides computer systems for performing the methods. The subject methods and computer systems find application in identifying modulators, i.e. enhancers or inhibitors, of protein-protein interactions, and, as such, find use in a variety or medical and research applications, including drug discovery.

In many embodiments, the subject invention is a software tool that identifies short peptides corresponding to sites that participate in protein-protein interactions by analyzing the primary sequence of a protein. Accordingly, the subject methods may be used to predict sites of protein-protein interaction in a wide variety of proteins because the methods do not rely on a known structure, experimental data, or even the identity of a binding partner.

In many cases, peptides identified by the subject methods have the ability to interfere with specific protein-protein interactions. Accordingly, the subject methods provide novel pharmacological tools to investigate the mechanism of action for proteins of interest, and aid in the process of drug discovery. For example, peptides identified by the subject methods can act as specific inhibitors by blocking a protein-protein interaction between an enzyme and its protein binding partner or as activators by interfering with an intramolecular protein-protein interaction in a manner that renders a protein constitutively active. Peptides identified by the subject methods are usually highly specific and able to distinguish between homologous proteins in the same family.

### DEFINITIONS

Unless defined otherwise, all technical and scientific terms used herein have the same meaning as commonly understood by those of ordinary skill in the art to which this invention pertains. Although any methods and materials similar or equivalent to those described herein can be used in the practice or testing of the present invention, the preferred methods and materials are described. The following definitions are provided to assist the reader in the practice of the invention.

The terms “polypeptide” and “protein” are used interchangeably throughout the application and mean at least two covalently attached amino acids, which includes proteins, polypeptides, oligopeptides and peptides. The protein may be made up of naturally occurring amino acids and peptide bonds, or synthetic peptidomimetic structures. Thus “amino acid”, or “peptide residue”, as used herein means both naturally occurring and synthetic amino acids. For example, homo-phenylalanine, citrulline and noreleucine are considered amino acids for the purposes of the invention. “Amino acid” also includes imino acid residues such as proline and hydroxyproline. The side chains may be in either the (R) or the (S) configuration. Normally, the amino acids are in the (S) or L-configuration. If non-naturally occurring side chains are used, non-amino acid substituents may be used, for example to prevent or retard in vivo degradation. Naturally occurring amino acids are normally used and the protein is a cellular protein that is either endogenous or expressed recombinantly. A “peptide” is a polypeptide that is about 3 to 50 amino acids in length, usually about 5-20 amino acids in length.

By “nucleic acid” herein is meant either DNA or RNA, or molecules which contain both deoxy- and ribonucleotides.

The term “computer readable medium” as used herein refers to any storage or transmission medium that participates in providing instructions and/or data to a computer for execution and/or processing. Examples of storage media include floppy disks, magnetic tape, CD-ROM, a hard disk drive, a ROM or integrated circuit, a magneto-optical disk, or a computer readable card such as a PCMCIA card and the like, whether or not such devices are internal or external to the computer. A file containing information may be “stored” on computer readable medium, where “storing” means recording information such that it is accessible and retrievable at a later date by a computer.

With respect to computer readable media, “permanent memory” refers to memory that is permanent. Permanent memory is not erased by termination of the electrical supply to a computer or processor. Computer hard-drive ROM (i.e. ROM not used as virtual memory), CD-ROM, floppy disk and DVD are all examples of permanent memory. Random Access Memory (RAM) is an example of non-permanent memory. A file in permanent memory may be editable and re-writable.

In certain embodiments of the invention, polypeptide sequences may be entered into a computer by “entering text”. Text may be entered using any known method, including typing text (e.g., using a keyboard or mouse or copy and pasting) into a user interface displaying a file, typing text directly into a file, or importing text from a spreadsheet, etc

The term “using” is used herein as it is conventionally used, and, as such, means employing, e.g., putting into service, a method or composition to attain an end. For example, if a program is used to predict a binding site, a program is executed to make a file, the file usually being the output of the program containing the sequence of the binding site. In another example, if an algorithm is used, it is usually accessed, read, and the information stored in the file employed to attain an end. Similarly if a unique identifier, e.g. a barcode is used, the unique identifier is usually entered to identify, for example, an object or file associated with the unique identifier.

Other definitions of terms appear throughout the specification.

Before the present invention is further described, it is to be understood that this invention is not limited to particular embodiments described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are now described. All publications mentioned herein are incorporated herein by reference to disclose and describe the methods and/or materials in connection with which the publications are cited.

It must be noted that as used herein and in the appended claims, the singular forms “a,” “and,” and “the” include plural referents unless the context clearly dictates otherwise. Thus, for example, reference to “a homolog” includes a plurality of homologs and reference to “the isozyme” includes reference to one or more such isozymes and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

Many of the biochemical and molecular biology methods referred to herein are well known in the art, and are described in, for example, Sambrook et al., Molecular Cloning: A Laboratory Manual, Cold Spring Harbor Press, New York Second (1989) and Third (2000) Editions, and Current Protocols in Molecular Biology, (Ausubel, F. M., et al., eds.) John Wiley & Sons, Inc., New York (1987-1999).

Methods For Predicting A Site of Protein-Protein Interactions

The invention provides methods for predicting a site of protein-protein interactions in a protein that involves aligning two homologous polypeptides and identifying a window that is significantly different between the two polypeptides. In many embodiments, the methods are performed by a computer, however, in some embodiments, the methods may be performed by manually (i.e., without the aid of a computer).

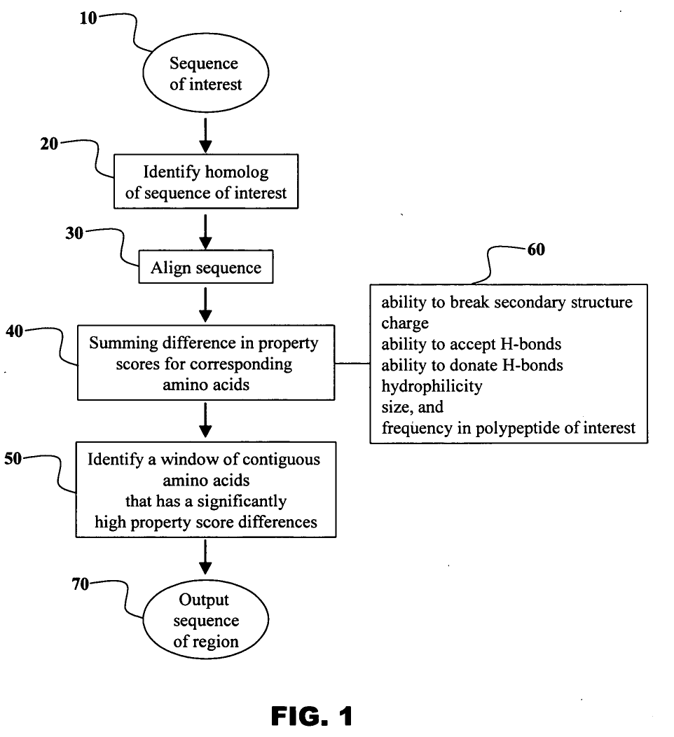

The invention is most easily described with reference to the flow diagram set forth in FIG. 1. The flow diagram set forth in FIG. 1 exemplifies the subject methods, and should not be used to limit the claimed invention.

As a first step in the method and with reference to FIG. 1, a polypeptide of interest is chosen 10. The sequence of interest may be any polypeptide, or fragment thereof. For example, the polypeptide of interest may be the full length amino acid sequence of a protein deposited in a database, e.g., GenBank, or a fragment of this protein, where the fragment may be greater than about 10 contiguous amino acids, greater than about 20 contiguous amino acids, greater than about 50 contiguous amino acids, greater than about 100 contiguous amino acids or greater than about 200 or 500 contiguous amino acids or more. Accordingly, when a “polypeptide of interest” is recited herein, it is intended to encompass full length polypeptides of interest, as well as any fragment thereof. The polypeptide of interest may be suspected of being involved in a protein-protein interaction, e.g., it is predicted to have interaction domains based on experimental data (e.g., data from two hybrid assays), structural data (e.g., data obtained from crystal structure of a the polypeptide), computational data (e.g., data obtained from aligning two proteins to find similar regions), etc., or a combination thereof. In many embodiments, however, a polypeptide is chosen simply because it is of interest. The polypeptide of interest may be from any species of organism, including bacteria, viruses, yeast and fungi, plants, and animals, including mammals such as humans.

As a second step, a polypeptide that is homologous to the polypeptide of interest is identified 20. This “homologous” polypeptide is usually highly related to the polypeptide of interest and is usually greater than about 50% identical, greater than about 60% identical, greater than about 70% identical, greater than about 80% identical, greater than about 90% 30 identical, greater than about 95% identical, usually up to about 98% of 99% identical to the polypeptide of interest along the entire length of the shortest of the two polypeptides. As would be recognized by one of skill in the art, a polypeptide of interest and a polypeptide that is homologous to the polypeptide of interest may be represented by two “isozymes”. Isozymes are usually enzymes that have similar, identical, or near identical biochemical activities, and can only be distinguished using certain physical characteristics (e.g., electrophoretic characteristics) or by their structure (e.g., their primary amino acid sequence). In most cases, isozymes arose in evolution by gene duplication and their number increases as a function of distance on the evolutionary tree. For example humans have more PKC isozymes (11) than Drosophila (2), C. elegans (1) Aplysia (2), and yeast (1) (Manning et al. Science. 2002 Dec. 6; 298(5600):1912-34). Exemplary isozymes include the members of the protein kinase C (PKC) family, and members of the PKA, Ras, Raf, cytochrome P-450, glucose-6-phosphatase (G6Pase), and nitric oxide synthase families, and isozymes described in the kinome database (Manning et al. Science. 2002 298:1912-34).

Homologous peptides may be identified by searching literature, e.g., references deposited in the Pubmed/Medline database, or accessions deposited in Genbank for proteins similar to the protein of interest. For example, typing in the name of the protein of interest and the word “isozyme” will often identify another protein that is an isozyme of the protein of interest that has already been identified. If a homologous polypeptide is not already known, a homologous polypeptide may be identified using any one of a variety of different methods. For example, a homologous polypeptide may be identified by searching a database of polypeptide sequences to identify polypeptides that are similar in sequence to the polypeptide of interest. These database searching methods are well known, and may be performed using the BLAST algorithm, described in Altschul et al., J. Mol. Biol. 215, 403-410, (1990) and Karlin et al., PNAS USA 90:5873-5787 (1993). A particularly useful BLAST program is the WU-BLAST-2 program (Altschul et al., Methods in Enzymology, 266: 460-480 1996). These algorithms use several search parameters, most of which are set to the default values. If present, the adjustable parameters may be set with the following values: overlap span=1, overlap fraction=0.125, word threshold (T)=11. The HSP S and HSP S2 parameters are dynamic values and are established by the program itself depending upon the composition of the particular sequence and composition of the particular database against which the sequence of interest is being searched; however, the values may be adjusted to increase sensitivity. In order to avoid the identification of orthologs (i.e., related polypeptides from other species) these database searches should be restricted to polypeptides from the species from which the polypeptide of interest is derived. For example, if the polypeptide of interest is a human polypeptide, then the homolog of that polypeptide should also be human. However, homologous proteins should not be allelic variants (i.e., they should not be encoded by genes situated at the same position in the genome of two different individuals). Since allelic variants usually have very high levels of sequence identity, e.g., 98%, 99% or even 100% sequence identity, they are easily identified and eliminated. In many embodiments, a polypeptide that is most homologous (i.e., most similar), based on a P-value (i.e., a probability value) or a percent identity to the polypeptide of interest, is chosen, providing that polypeptide is not an allelic variant of and is from the same species as the polypeptide of interest. Accordingly, a polypeptide that is homologous to a polypeptide of interest may be identified.

The sequence of the polypeptide of interest and the sequence of the homologous polypeptide are then aligned 30. This alignment may be done by eye, i.e., visually comparing the two sequences and aligning them, however, as is known in the art, sequence alignment is most effectively done using one of many known algorithms for aligning sequences. For example, sequences may be aligned using standard techniques known in the art, including, but not limited to, the local sequence identity algorithm of Smith & Waterman (Adv. Appl. Math. (1981) 2:482), the sequence identity alignment algorithm of Needleman & Wunsch (J. Mol. Biol. (1970) 48:443), the search for similarity method of Pearson & Lipman, (PNAS USA (1988) 85:2444), the computerized implementations of these algorithms (GAP, BESTFIT, FASTA, and TFASTA, etc., as found in the Wisconsin Genetics Software Package, Genetics Computer Group, 575 Science Drive, Madison, Wis.), and the Best Fit sequence program described by Devereux et al., (Nucl. Acid Res. (1984) 12:387-395), using the default settings. In certain embodiments, an alignment of two polypeptides first employs a known alignment tool and is further refined by eye, for example, by creating and moving gaps.

The alignment of the sequences of two polypeptides identifies corresponding amino acids, where “corresponding” amino acids are amino acid residues that are positioned across from each other when the two sequences are aligned. Accordingly, the term “corresponding” defines an amino acid by its positional relationship with an amino acid in a different polypeptide when the two polypeptides are aligned. In other words, the amino acid in a homologous polypeptide that corresponds to a particular amino acid in a polypeptide of interest lies across from that amino acid when the sequences of the two polypeptides are aligned. Corresponding amino acids may be the same amino acids or different amino acids.

Next, the differences in property scores between corresponding amino acids are summed 40. In general, a property score is a numerical assessment of a biochemical property of an amino acid and/or a frequency that the amino acid is present. In most embodiments, each of the 20 natural amino acids is characterized by a set of property scores that each numerically describe a different biochemical or statistical property. The ability to break secondary structure, charge, ability to accept H-bonds, ability to donate H-bonds, hydrophilicity and size of an amino acid 60 are biochemical properties of interest in the subject methods. Frequency is a statistical property.

There are many ways of scoring the biochemical properties of amino acids and the exact numbering system (i.e., the scale used) used may be arbitrary chosen. For example, a binary scoring system, e.g., “0” and “1”, may be used to indicate some biochemical properties e.g., H-bond accepting potential (where “0” indicates no potential and “1” indicates significant potential). Other scoring systems may also be used, e.g., “0”, “1” and “2” etc. For example, some biochemical properties, e.g., charge may be assessed on a “0”, “1” and “2” scale (where “0” is low or no charge, “1” is negative charge and “2” is positive charge). An exemplary scoring system is set forth in Table 1.

As discussed above, one of the property scores indicates the frequency that an amino acid is present in the polypeptide of interest, or, in other embodiments, in a plurality of polypeptides, such as those in a database. If an amino acid is rare, e.g. trp, the amino acid may be scored highly, e.g. “1” on a binary scale, or “2” on a “0”, “1” and “2” scale. For example, for following scoring system may be used: W=0.01, Y=0.03, M=0.03, C=0.03, C=0.03, H=0.03, N=0.04, Q=0.04, T=0.4, A=0.05, I=0.05, R=0.05, P=0.06, S=0.06, S=0.06, F=0.06, V=0.06, D=0.06, E=0.07, G=0.07, K=0.07, L=0.08 (these numbers represent the amino acid usage in all PKCs in mouse). Amino acid frequencies are easily calculated and readily incorporated into the subject methods.

To calculate the difference in property scores for a pair of corresponding amino acids, the differences between the individual properties scores of those amino acids are summed. In other words, the differences in each property score for two corresponding amino acids is first calculated, and then these differences are added together. For example, for a pair of corresponding amino acids, if only two biochemical properties are assessed, e.g., a) charge, and b) H-bond accepting potential, then the amino acids are assigned a first score indicating their charge, and a second score indicating their H-bond accepting potential. The difference in property scores between the amino acids is then calculated for each property, and the differences between the properties scores are summed. This process can be used to calculate a summed property score difference between any two amino acids. An example of calculating the summed property score difference between leucine and tyrosine is shown in Table 2.

Accordingly, each pair of corresponding amino acids may be assigned a summed difference in property scores, which, in most embodiments, represents a numerical assessment of how different the amino acids are to each other. These property score differences are usually expressed as a sequence of numbers, corresponding to the contiguous sequence of amino acids analyzed. An example of such a sequence of property scores differences may be seen in Table 3. The property scores are termed “value difference” in this table.

The next step in these methods is to identify a window of contiguous amino acids that has significantly high property score differences 50. This step is usually done by scanning the sequence of property score differences to find a “window”, e.g., a region of at least 5, at least 6, at least 7, at least 8, at least 9, at least 10 (e.g., 11, 12, 13, etc.) or more contiguous property scores that is above or equal to a threshold difference. This threshold difference may be calculated by any one of a number of means. Similar means have been used to calculate antigenic indices of polypeptides, and generally involve “tiling”, i.e., a window of a certain size that moves, one residue at a time, along a polypeptide (e.g., Hopp and Woods, (1981) Proc Natl Acad Sci USA 86:152-156).

Most polypeptides have one, two, three, four or five windows that have property score differences above the threshold difference. As is discussed below, the threshold difference may be identified using any one of a number of different methods.

In one embodiment, the threshold difference is represented by the window of property score differences having the highest differences. In these embodiments, a window is moved along the length of the sequence of property score differences, and at each window position the differences in property scores within the window is assessed. As would be recognized by one of skill in the art, the property score differences within the window could be averaged, or summed, etc., to provide this assessment. Accordingly, for one polypeptide a window with the highest property score differences may be identified, and the difference in property scores associated with this window (e.g., the summed differences or average thereof) may be used as the threshold difference. In this embodiment, each polypeptide has a single region having significantly high property score differences. This region is represented by the window having the highest property score differences.

In another embodiment, a threshold difference is represented by a fraction of non-overlapping windows that have the highest differences in property scores. In these embodiments, as above, a window is moved along the length of the sequence of property scores, and at each window position the differences in property scores in the window is assessed. The threshold difference may be obtained from the windows having the highest property score differences. For example, the threshold difference may be obtained from a percentage (e.g. 10%, 20%, etc.) of windows with the highest property score differences. In other words, the threshold difference is a difference in property scores that distinguishes between the windows with low property score differences and those with high property score differences by calculating the lowest property score differences for the windows with the highest property score differences (e.g., the top 10%, 20%, etc. of windows having the highest property score differences). In this embodiment, each polypeptide may have more than one region having significantly high property score differences, depending on the number desired.

As would be readily apparent, a number of other statistical methods may be used calculate threshold values. For example, a coefficient of variation analysis of the property score differences for all windows of a polypeptide would reveal windows with property score differences over a certain threshold (e.g., greater or less than one or two standard deviations from a mean window property score differences, etc.).

Threshold differences may also be determined prior to analysis of a protein of interest. Since any numbering scheme for assessing property score differences may be used, the threshold difference may very widely. In many embodiments, however, if pairs of amino acids can be generally separated in to three categories, according to their differences property score: “same” (where the amino acids in the pair are identical), “similar” (where the amino acids in the pair are similar, e.g., “conserved”) and “different” (where the amino acids in the pair are different, i.e., not conserved or the same). If a window contains property score differences that are above a threshold difference, then all or most of the amino acids pairs within a window are different. For example, using a simple property score numbering system using the numbers “0”, “1” and “2”, indicating the same, similar and different amino acids, a window having a significant proportion of amino acids having property score differences of greater than or equal to 2 may represent a window with a difference in property scores that is greater than a threshold difference. For example, in the embodiment set forth below, any window of 6 property differences that has at least 5 property differences of greater than 2 is above a threshold difference. Of course, depending on the size of the window and the numbering system, the threshold difference may change.

In many embodiments, after a window that has a property score differences that are greater than a threshold difference is identified, the window is expanded to encompass property score differences that are greater than the original window. In many embodiments, the window is expanded until it reaches a pre-determined property score difference, e.g., a property score of “0” (i.e. identical amino acids), or two or three consecutive property scores of “0”, etc.

By identifying a window of property score differences that is above a threshold difference, a region of at least 6 contiguous amino acids that have significantly high property score differences may be identified. As a final step in this process the sequence of the amino acids of this region is identified, and, in some embodiments exported 70.

As mentioned above, this method may be performed by hand or, in many embodiments, using a computer. In computer-related embodiments, the methods described above are in the form of an algorithm, or programming, for performing the methods. In computer-based methods, there is usually an input for inputting a sequence of interest into the memory of a computer, and an output, that displays or exports the amino acid sequence of a predicted site of protein-protein interaction. In many computer based methods a database of property scores which assigns a numerical score to each of several properties of each amino acid, such as that described in Table 1, is employed. Computer based methods may also contain a database of amino acid sequences, and an algorithm for identifying similar homologous polypeptides, such as a BLAST algorithm.

In some embodiments, the computer based methods require entry or selection of a sequence of interest and a polypeptide homologous to the sequence of interest, and execution of an algorithm. In other embodiments, the computer may have a means for automatically identifying homologous polypeptides, and, accordingly, the computer based methods may require entry or selection of a sequence of interest, and execution of an algorithm. In most embodiments, the output of the algorithm will be a sequence of amino acids that corresponds to a region of a polypeptide of interest that is significantly different to a corresponding region in a polypeptide that is homologous to the polypeptide of interest. In many embodiments, the output may be a file, e.g., a table, and the file may be stored in the memory of a computer.

As mentioned above, the invention provides methods of designing peptide modulators, i.e. inhibitors or enhancers of protein-protein interaction. In general, these methods involve predicting a site of protein-protein interactions using the methods set forth above, and designing a peptide (i.e., a proteinaceous compound having about 5-50 amino acids or mimetics thereof), that contains the predicted site. These modulatory peptides may designed, manufactured, and used to modulate, e.g., inhibit protein-protein interactions of the polypeptide of interest. Methods for modulating protein-protein interactions may be done in vitro, in isolated or cultured cells, using isolated organs ex vivo or in vivo. In many embodiments, the peptide may be conjugated to a carrier moiety, such as TAT peptide, antennapedia peptide or polyarginine, to facilitate entry of the peptide into a cell.

For example, a polypeptide of interest is chosen, e.g., a polypeptide that is involved in cellular signaling whose activity is desirable to modulate, and a site of protein-protein interaction is predicted on the polypeptide using the methods described above. A peptide is then made containing the same amino acids as the predicted site (or analogs thereof), in the same order as the predicted site. The peptide may be longer than the predicted site, usually by at least 2, at least 5, or at least 10 amino acids and may be designed or modified to have increased solubility, stability and circulating time of the polypeptide, or decreased immunogenicity (see U.S. Pat. No. 4,179,337). For example, the peptide may be derivatized by a water soluble polymer such as polyethylene glycol, ethylene glycol/propylene glycol copolymers, carboxymethylcellulose, dextran, polyvinyl alcohol and the like.

After synthesis, the polypeptide may be introduced into a cell and a cellular phenotype (e.g., gene expression, intracellular calcium levels, marker expression, etc.) assessed. The cellular phenotype, of course, varies depending on the identity of the polypeptide of interest. In most embodiments, the peptide will reduce binding of the polypeptide of interest to a binding partner and modulating the activity of the polypeptide of interest (e.g., inhibit cellular signaling). The synthesized peptide usually reduces or increases binding of the polypeptide of interest to at least one binding partner by at least 10%, at least 20%, at least 40%, at least 60%, at least 80%, at least 90%, or, in some embodiments, 95% or more, usually up to 99% or 100% to increase or reduce a cellular phenotype by a similar amount, or more.

Accordingly, peptides designed using the subject methods find use agents for modulating protein-protein interactions in a cell. Since several diseases and conditions, e.g., several cancers, inflammatory diseases, and chronic diseases, have altered protein-protein interactions the subject peptides find use as potential treatments for a vast variety of medical conditions.

Programming, Computer Readable Media And Computer Systems

The subject invention provides computer programming written on computer readable media for performing the methods set forth above. While the subject programming finds use in a variety of settings, it is most commonly used in a computer system comprising a processor, a memory, an input, and an output that are coupled to each other.

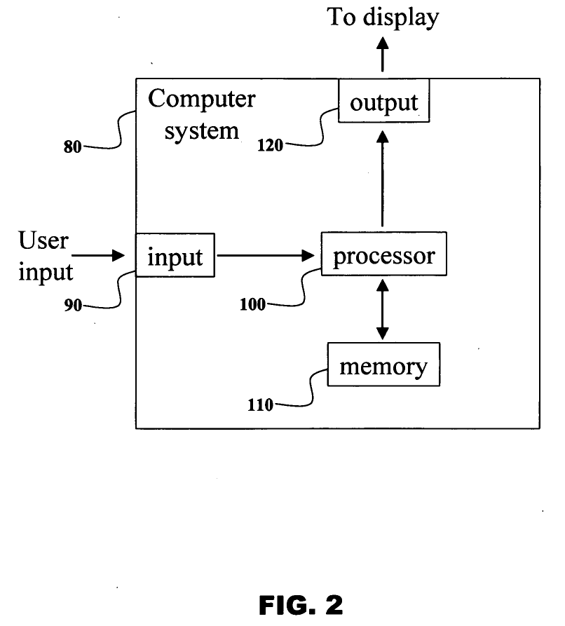

FIG. 2 is a simplified block diagram of computer system 80 according to an embodiment of the present invention. Computer system 80 typically includes at least one processor 100 which communicates with a number of peripheral devices. These peripheral devices typically include a memory 110, a user interface input device 90, user interface output device 120 (e.g. a monitor). The input and output devices allow user interaction with computer system 80. It should be apparent that the user may be a human user, a device, another computer, and the like.

User interface input devices 90 may include a keyboard, pointing devices such as a mouse, trackball, touchpad, or graphics tablet, a scanner, a touchscreen incorporated into the display, audio input devices such as voice recognition systems, microphones, and other types of input devices. In general, use of the term “input device” is intended to include all possible types of devices and ways to input information into computer system 80.

User interface output devices 120 may include a display subsystem, a printer, a fax machine, or non-visual displays such as audio output devices. The display subsystem may be a cathode ray tube (CRT), a flat-panel device such as a liquid crystal display (LCD), or a projection device. The display subsystem may also provide non-visual display such as via audio output devices. In general, use of the term “output device” is intended to include all possible types of devices and ways to output information from computer system 80 to a human or to another machine or computer system.

Memory 110 stores the basic programming and data constructs that provide the functionality of the various systems embodying the present invention. For example, algorithms for performing the methods set forth above may be stored in memory 110. These software modules are generally executed by processor 100. In a distributed environment, the software modules may be stored on a plurality of computer systems and executed by processors of the plurality of computer systems. Memory 110 also provides a repository for storing the various databases storing information according to the present invention.

Memory 110 typically includes a number of memories including a main random access memory (RAM) for storage of instructions and data during program execution and a read only memory (ROM) in which fixed instructions are stored. A file storage subsystem may provide persistent (non-volatile) storage for program and data files, and usually includes a computer readable media, e.g., a hard disk drive, a floppy disk drive along with associated removable media, a Compact Digital Read Only Memory (CD-ROM) drive, an optical drive, removable media cartridges, and other like storage media. One or more of the drives may be located at remote locations on other connected computers at another site on a communication network.

Computer system 80 itself can be of varying types including a personal computer, a portable computer, a workstation, a computer terminal, a network computer, a television, a mainframe, or any other data processing system. Due to the ever-changing nature of computers and networks, the description of computer system 80 depicted in FIG. 2 is intended only as a specific example for purposes of illustrating a common embodiment of the present invention. Many other configurations of a computer system are possible having more or less components than the computer system depicted in FIG. 2.

Kits

Kits for use in connection with the subject invention may also be provided. Such kits usually include at least a computer readable medium including programming as discussed above and instructions. The instructions may include installation or setup directions. The instructions may include directions for use of the invention with options or combinations of options as described above. In certain embodiments, the instructions include both types of information. In some embodiments, the programming contains a database of amino acid property scores, a database of pairs of homologous polypeptides (e.g., isozymes), and the like.

Providing the software and instructions as a kit may serve a number of purposes. The combination may be packaged and purchased as a means of upgrading feature extraction software. Alternately, the combination may be provided in connection with new software. In many embodiments, the instructions will serve as a reference manual (or a part thereof) and the computer readable medium as a backup copy to the preloaded utility.

The instructions are generally recorded on a suitable recording medium. For example, the instructions may be printed on a substrate, such as paper or plastic, etc. As such, the instructions may be present in the kits as a package insert, in the labeling of the container of the kit or components thereof (i.e., associated with the packaging or subpackaging), etc. In other embodiments, the instructions are present as an electronic storage data file present on a suitable computer readable storage medium, e.g., CD-ROM, diskette, etc, including the same medium on which the program is presented.

In yet other embodiments, the instructions are not themselves present in the kit, but means for obtaining the instructions from a remote source, e.g. via the Internet, are provided. An example of this embodiment is a kit that includes a web address where the instructions can be viewed and/or from which the instructions can be downloaded. Conversely, means may be provided for obtaining the subject programming from a remote source, such as by providing a web address. Still further, the kit may be one in which both the instructions and software are obtained or downloaded from a remote source, as in the Internet or world wide web. Some form of access security or identification protocol may be used to limit access to those entitled to use the subject invention. As with the instructions, the means for obtaining the instructions and/or programming is generally recorded on a suitable recording medium.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1

### Summary of Pfinder

The following examples describe an example of the invention called “pFinder”. This example is provided to exemplify, and not to limit, the invention claimed herein.

The algorithm for pFinder is based on a rationale that sequences that are the least similar between the two isozymes are likely to mediate isozyme-specific protein-protein interactions. Accordingly, the interacting domains of two isozymes with a high degree of homology are compared. In addition to similarity, differences between aligned regions can be ranked according to their significance (i.e. the likelihood that the region participates in protein-protein interactions). This algorithm was used to identify three active peptides corresponding to unique regions in the V1 domain of δ and εPKC (protein kinase C). We also identified peptides derived from the V5 regions of βI and βII PKC that serve as selective inhibitors of each isozyme.

To identify regions that are the most likely to participate in protein-protein interactions, pFinder compares two aligned protein primary sequences and generates a numeric value corresponding to the significance of the differences between each amino acid pair. Higher numbers indicate more significant differences. For example, if two alanines (A) are aligned (and therefore are conserved between the two isozymes), pFinder assigns this pair a difference value of 0. In the case of two very different amino acids such as a lysine (K) aligned with an alanine (A), pFinder assigns this pair a difference value of 17. In the case of two similar amino acids, such as aspartic acid (D) and glutamic acid (E), the difference value is 1. These numerical values assigned to the differences between amino acids are based on the features and weights described below.

pFinder takes as input the primary sequence of the protein of interest and outputs a list of peptides that inhibit protein-protein interactions of the target protein. The user of pFinder also has the option of giving additional input that may augment pFinder's algorithm, for example a particular domain known to participate in a protein-protein interaction of interest. pFinder may interface with protein databases to extract information such as homologues or any known structures, and will incorporate all methods of rational design used by us to identify peptides. pFinder is a tool for peptide prediction for a wide range of proteins, including proteins without a known structure or binding partner.

### Example 2

### Assignment of Property Scores

pFinder 1.0 examines seven amino acid features (see Table 1). Six features correspond to the biochemical properties of the amino acids: ability to break secondary structure, charge, ability to accept H-bonds, ability to donate H-bonds, hydrophilicity and size. The seventh feature corresponds to a statistical property: frequency in a database. Specifically, how often does a particular amino acid appear in the protein that is being analyzed by the software (e.g. PKC).

These features were chosen both by examining previously identified peptide modulators as well as knowledge based reasoning about what features are important in protein-protein interactions. For example, there are many published values for biochemical properties of amino acids as well as amino acid feature matrices, including those documented in the AAIndex Database. We used amino acid biochemical data to build pFinder's features matrix. For example, to describe the size of the amino acids, we averaged the surface area and volume of the twenty amino acids. We then ranked them and separated them into three groups (small, medium, and large).

Each feature is represented by numerical values. For many features, a value of 1 indicates that the amino acid has that particular feature and a value of 0 indicates the lack of that feature. For example, an amino that has the potential to form H-bonds via its side chain has the value of 1 for this feature (e.g., cysteine).

Other features have three values, 0, 1 and 2. For some features, the three values (0, 1 and 2) indicate three levels of that feature. For example, for the size feature a value of 0 corresponds to small amino acids, 1 for medium amino acids, and 2 for large. For this category of features, the difference between a value of 0 and a value of 2 is greater than the difference between a value of 1 and a value of 2, or a value of 0 and a value of 1.

The three values 0-2 can also represent three states. One example is the feature charge, for which a value of 0 indicates neutral, 1 for negatively charged amino acids, and 2 for positively charged amino acids. In this case, the difference between a negatively charged amino acid (with a value of 1) and a neutral amino acid (with a value of 0) is not necessarily smaller than the difference between a positively charged amino acid (with a value of 2) and a neutral amino acid.

The values scores for each property for each amino acid used in pFinder are shown in Table 1.

TABLE 1
Amino acid features used by pFinder 1.0 to characterize important differences between two amino
acids. Charge, hydrophilicity and size are given the most weight.

| | Feature/Amino Acid | | | | | | | | | | | | | | | | | | | |
|---|---|---|---|---|---|---|---|---|---|---|---|---|---|---|---|---|---|---|---|---|
| | A | C | D | E | F | G | H | I | K | L | M | N | P | Q | R | S | T | V | W | Y |
| Charge | 0 | 0 | 1 | 1 | 0 | 0 | 2 | 0 | 2 | 0 | 0 | 0 | 0 | 0 | 2 | 0 | 0 | 0 | 0 | 0 |
| H-bond accepting | 0 | 0 | 1 | 1 | 0 | 0 | 1 | 0 | 0 | 0 | 0 | 1 | 0 | 1 | 0 | 1 | 1 | 0 | 0 | 1 |
| potential | | | | | | | | | | | | | | | | | | | | |
| H-bond donating | 0 | 1 | 0 | 0 | 0 | 0 | 1 | 0 | 1 | 0 | 0 | 1 | 0 | 1 | 1 | 1 | 1 | 0 | 1 | 1 |
| potential | | | | | | | | | | | | | | | | | | | | |
| Hydrophilicity | 0 | 0 | 1 | 1 | 0 | 0 | 1 | 0 | 1 | 0 | 0 | 1 | 1 | 1 | 1 | 1 | 1 | 0 | 0 | 1 |
| Rarity | 0 | 1 | 0 | 0 | 0 | 0 | 1 | 0 | 0 | 0 | 1 | 0 | 0 | 0 | 0 | 0 | 0 | 0 | 2 | 1 |
| Secondary Structure | | | | | | | | | | | | | | | | | | | | |
| Breaker | 0 | 0 | 0 | 0 | 0 | 1 | 0 | 0 | 0 | 0 | 0 | 0 | 1 | 0 | 0 | 0 | 0 | 0 | 0 | 0 |
| Size | 0 | 1 | 1 | 1 | 2 | 0 | 2 | 2 | 2 | 2 | 2 | 1 | 1 | 1 | 2 | 0 | 1 | 1 | 2 | 2 |

### Example 3

### Weighting of Property Scores And Calculating the Difference In Property Scores

pFinder weighs three features more heavily than the other features. These are charge, hydrophilicity and size. Therefore, two amino acids with a difference in these features will be given a higher numerical difference value. These weights were chosen both by examining previously identified peptide regions as well as knowledge-based reasoning about the relative importance of each feature.

The combination of features and weights allow pFinder to generate a numerical value for each amino acid pair that reflects how different the two amino acids are. For example, pFinder calculates the difference value for the amino acid pair leucine (L) and tyrosine (Y) by adding the weighted differences for each of the features (see Table 2).

TABLE 2
Calculating the numerical difference value for the amino acid pair
leucine and tyrosine. The sum of the weighted differences for each
of the features, plus 1 because the two are non-identical, results
in a numerical difference value of 9 for this pair. Note that
hydrophilicity is weighed more heavily than features such
as H-bonding potential.

| Feature/Amino Acid | L | Y | Weighted Difference |
|---|---|---|---|
| Charge | 0 | 0 | 0 |
| H-bond accepting potential | 0 | 1 | 1 |
| H-bond donating potential | 0 | 1 | 1 |
| Hydrophilicity | 0 | 1 | 5 |
| Rarity | 0 | 1 | 1 |
| Secondary Structure Breaker | 0 | 0 | 0 |
| Size | 2 | 2 | 0 |
| Sum: | | | 8 + 1 (non-identical) = 9 |

### Example 4

### Identifying Sites of Protein-Protein Interaction

pFinder's algorithm begins by choosing regions within a domain that have at least 5 out of 6 adjacent amino acid pairs that are not conserved. The one allowed conserved pair should not lie on the edge of the region. A peptide corresponding to this small region is chosen to be as long as possible while still fulfilling the constraint of no more than one conserved pair. pFinder's algorithm then further prunes any peptides that correspond to regions containing 50% or more numerical difference values that are less than or equal to 2. Amino acid pairs with these low numerical value scores correspond to homologous amino acids, and therefore are unlikely to specify a region that provides unique protein-protein interactions. Results of pFinder analysis of δPKC and θPKC are revealed in Table 3, below.

TABLE 3
The first 20 amino acids in the V1 domains of δPKC (SEQ ID NO: 1) and
θPKC(SEQ ID NO: 2). pFinder assigned difference values are indicated above each amino acid
pair. Higher numbers indicate a greater difference between the amino acids. Identical amino
acids have a difference value of 0. pFinder's algorithm located a peptide region, shown in bold
red, by identifying a sequence of at least 5 out of 6 significantly different adjacent amino acids.
This peptide correlates exactly to a previously identified peptide inhibitor, δV1-1.

| | Difference value | | | | | | | | | | | | | | | | | | | |
|---|---|---|---|---|---|---|---|---|---|---|---|---|---|---|---|---|---|---|---|---|
| | 0 | 10 | 0 | 0 | 0 | 0 | 0 | 11 | 7 | 6 | 6 | 9 | 1 | 11 | 0 | 5 | 11 | 0 | 0 | 0 |
| δPKC | M | A | P | F | L | R | I | S | F | N | S | Y | E | L | G | S | L | Q | A | — |
| θPKC | M | S | P | F | L | R | I | G | L | S | N | F | D | C | G | T | C | Q | A | C |

In addition to being designed for isozyme specificity, pFinder peptides may be designed to act as cargo with cell permeable peptide carriers such as TAT peptide, antennapedia-derived or polyarginine peptides. This may be accomplished by providing a cysteine residue, which allows for the formation of a cysteine S—S bond between carrier and cargo. Thus pFinder peptides are pharmacological agents that are able to enter into cells.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

1. A method for predicting a site of protein-protein interaction, comprising:
calculating the difference in property scores between amino acids of a corresponding pair of amino acids on two homologous polypeptides; and
identifying a window of consecutive amino acids that has a difference in property scores that is greater than a threshold difference;
to predict a site of protein-protein interaction.

2. The method of claim 1, wherein said method is a computer based method.

3. The method of claim 1, wherein said two homologous polypeptides are isozymes.

4. The method of claim 1, wherein said property scores are an assessment of:
at least one biochemical property of an amino acid; and
the frequency that said amino acid appears in one of said homologous polypeptides.

5. The method of claim 4, wherein said biochemical properties are:
ability to interrupt secondary structure;
charge;
ability to accept H-bonds;
ability to donate H-bonds;
hydrophilicity; and
size,
of an amino acid.

6. The method of claim 5, wherein at least one of said properties is weighted as compared to other properties.

7. The method of claim 1, wherein said property score is a numerical value and said difference represents a difference in said numerical values.

8. The method of claim 1, further comprising inputting sequences for said homologous polypeptides into the memory of a computer.

9. The method of claim 1, wherein said window is a window of at least six contiguous amino acids.

10. The method of claim 1, wherein said site of protein-protein interaction is a intermolecular or intramolecular site of protein-protein interaction.

11. A computer system for predicting a site of protein-protein interaction, comprising:
a processor;
a memory coupled to the processor, the memory configured to store instructions for execution by the processor, the instructions comprising:
instructions for inputting amino acid sequences of two homologous polypeptides;
instructions for calculating the difference in property scores between amino acids of a corresponding pair of amino acids of said two homologous polypeptides;
instructions for identifying at least six contiguous amino acids that have a difference in property score that is greater than threshold difference,
instructions for outputting the amino acids sequence of said at least six contiguous amino acids,
wherein said output amino acid sequence is predicted to be a site of protein-protein interaction.

12. The computer system of claim 11, wherein property scores for each amino acid are stored in a database

13. The computer system of claim 11, further comprising a user interface for inputting an amino acid sequence.

14. The computer system of claim 13 wherein said user interface provides for selection of a pre-established file.

15. The computer system of claim 13, wherein said user interface provides for direct entry of a sequence into said interface.

16. An computer readable medium comprising instructions for performing the method of claim 1.

17. A kit comprising the computer readable medium of claim 16.

18. A method of designing a peptide modulator of a protein-protein interaction, comprising,
calculating the difference in property scores between amino acids of a corresponding pair of amino acids on two homologous polypeptides; and
identifying at least six contiguous amino acids that have a difference in property scores that is greater than a threshold difference;
to design a peptide modulator of a protein-protein interaction.

19. A method for producing a peptide modulator of a protein-protein interaction, comprising,
designing a peptide modulator of a protein-protein interaction according to the method of claim 18, and
manufacturing said peptide modulator.

20. A method for modulating a protein-protein interaction of a polypeptide, comprising:
producing a peptide modulator of a protein-protein interaction using the method of claim 19, and
contacting said peptide modulator with one of said homologous polypeptides, to modulator a protein-protein interaction of said polypeptide.


SEQUENCE LISTING (2 sequences)

SEQ ID NO: 1 - protein, 19 aa, H. sapien
MAPFLRISFNSYELGSLQA

SEQ ID NO: 2 - protein, 20 aa, H. sapien
MSPFLRIGLSNFDCGTCQAC